(19) United States
(12) Patent Application Publication
(10) Pub. No.: US 2012/0240941 A1
(43) Pub. Date: Sep. 27, 2012

(54) METHODS AND SYSTEMS FOR MONITORING SLEEP POSITIONS

(75) Inventors: Daniel C. Rosenman, San Francisco, CA (US); Mehran Farid-Moayer, Burlingame, CA (US)

(73) Assignee: Restorics, Inc., Burlingame, CA (US)

(21) Appl. No.: 13/425,093

(22) Filed: Mar. 20, 2012

Related U.S. Application Data
(60) Provisional application No. 61/466,389, filed on Mar. 22, 2011.

(51) Int. Cl. A61F 5/56; A61C 5/14; A61B 5/11

(52) U.S. Cl. 128/848

(57) ABSTRACT

A patient position monitor comprises an oral appliance and an electronic circuit module. The oral appliance is adapted to be held in a fixed location within the patient's mouth, typically being removably secured to the patient's teeth. Orientation circuitry detects patient's position and sends an alarm to the patient when the patient assumes a supine position and is at particular risk of snoring or sleep apnea.

### CROSS-REFERENCE

This application claims the benefit of provisional application No. 61/466,389 (Attorney Docket No. 42020-703.101), filed on Mar. 22, 2011, the full disclosure of which is incorporated hereby by reference.

### BACKGROUND OF THE INVENTION

1. Field of the Invention

The present invention relates to medical devices and methods. In particular, the present invention comprises an oral device and methods for its use in monitoring the sleep position of a patient, for example in treating sleep apnea and snoring.

Sleep apnea and snoring are common problems. While sleeping, muscles in the tongue and the soft palate relax and lose their tone. Loss of muscle tone in the tongue and soft palate can result in closure or narrowing of the pharyngeal airway which in turn can cause snoring and, in the case of complete closure of the airway, sleep apnea. Snoring is an annoyance and sleep apnea is a health risk associated with repetitive drops in blood oxygen saturation and/or arousal of the brain. Obstructive sleep apnea may further lead to cardiovascular complications and can also impair cognition, memory, and cause daytime sleepiness and fatigue.

Most current treatments rely on positive airway pressure therapy, surgical interventions or use of an oral appliance to maintain airway patency. Surgical interventions suffer from common problems associated with general surgery and have low success rates. Positive airway pressure therapy require wearing a mask and using a pressure pump that is effective, but is poorly tolerated by a group of patients. Oral appliances are well accepted and tolerated, but have less success rate in treating sleep apnea. Surgical treatments and oral appliances are more effective while the patient sleeps on his/her side.

An alternative or adjunctive treatment for helping patients stop snoring and/or avoid the complications of sleep apnea relies on preventing patients from sleeping on their backs or positions that worsen their sleep apnea. It is known that snoring and sleep apnea occur more frequently when a patient sleeps on his or her back, and these disorders are often avoided when the patient sleeps on his or her side or front. For example, it has been proposed to place a tennis ball or other light but bulky object on the back of the patient's sleep shirt. Should the patient roll over onto his or her back during the night, the object will cause discomfort and the patient will turn.

Other somewhat more sophisticated sleep position monitoring systems have been proposed. Patent applications describing a number of these systems are listed below in the Description of Background Art. Of these, perhaps the most relevant is U.S. Pat. No. 4,617,525, which describes a position monitor which is strapped to the patient's posterior torso and which provides an audible signal when the patient lies on his or her back for more than a predetermined threshold time. While likely effective, this alarm design would be poorly tolerated by many patients. The strapping configuration would be uncomfortable, and the sensor module location on the torso would have many of the discomforts associated with the more simple “tennis ball” design discussed previously. Additionally, the inclusion of an audible alarm would be an annoyance for anyone sleeping in proximity with the patient.

For these reasons, it would be desirable to provide improved apparatus and methods for monitoring sleep position of a patient which are useful for patients suffering from snoring and/or sleep apnea. It would be particularly desirable if the devices and methods were well tolerated by patients, provided minimum disruption of the patients' sleep at times other than when the patient is sleeping on his or her back, and which provide a clearly audible or discernible alarm to the patient while providing minimum or no disturbance to adjacent persons. At least some of these objectives will be met by the inventions described here and below.

2. Description of the Prior Art

Sleep position alarms are described in U.S. Pat. Nos. 4,617525; 5,038,137; 5,081,447; and 6,057,767. Other sleep alarms are described in U.S. Pat. Nos. 7,160,252; 7,141,020; 7,115,092; 7,087,027; 5,477,867; 5,123,425; 4,765,340; and 4,644,330 and Published Application Nos. 2008/269583; 2008/190436; 2008/243017; 2007/282212; 2007/172730; and 2006/064037.

All publications, patents, and patent applications mentioned in this specification are herein incorporated by reference to the same extent as if each individual publication, patent, or patent application was specifically and individually indicated to be incorporated by reference.

### SUMMARY OF THE INVENTION

The present invention provides improved methods and apparatus for monitoring the position of a patient during sleep. In particular, the methods and apparatus monitor orientation of the patient and alert the patient when the patient has turned onto his or her back (referred to as a “supine” position) for a time longer than a minimum threshold period, typically in the range from 30 seconds to 60 minutes often in the range from 1 minute to 5 minutes. The alarm causes the patient to turn away from the supine position which limits the likelihood of snoring and/or sleep apnea. In a group of patients sleeping on the side may be the disadvantageous position. In that case the current device could be programmed to promote sleeping in an advantageous position that is determined by the patient or his healthcare professional”.

The methods of the present invention typically rely on placement of an oral appliance within the patient's oral cavity. The oral appliance will be secured within the oral cavity to maintain a constant orientation relative to the patient's head. Position of the head is more relevant to sleep apnea and snoring than the position of the body. Indeed, snoring and sleep apnea occur most frequently when the head is oriented vertically (with the face oriented upwardly). Thus, the tracking of the position of the head serves as a direct measure of the likelihood of the occurrence of snoring and sleep apnea.

The oral appliance will usually be removably placed onto a firm or hard surface in the oral cavity, typically being irremovably secured over a tooth or group of teeth. The appliance, however, could alternatively be placed in other locations, such as on the hard palate, the jaw bone, or the like. In other cases, however, it may be possible to mount the device more permanently on either a hard or soft surface within the oral cavity. But such “permanent” placement will usually be less desirable.

The alarm provided by the methods and apparatus of the present invention will typically be audible or vibrational, but could also have a variety of other discernible outputs, such as electrical current, creating a taste, creating an aroma, or the like. In some instances, the alarm could represent a combination of these discernible events. In the case of sound (audible) and vibration, the sound and vibration may be transmitted to the cochlea via bone conduction. In all cases, the alarm which is generated will preferably be imperceptible to anyone else sleeping near the patient.

In a first aspect of the present invention, the method for monitoring sleep position of the patient comprises providing a module, having an alarm responsive to the module's orientation. The module is secured within the patient's cavity and provides an alarm discernible by the patient when the patient lies on his or her back, typically for a time period exceeding some minimum threshold time.

Most simply, the alarm may be responsive to changes in orientation relative to the gravitational field which surrounds the patient. Such gravitational field monitoring may be accomplished by a variety of conventional circuits, typically involving accelerometers, more typically two or three accelerometers mounted orthogonally relative to each other. While gravitational orientation will typically be used, other orientation monitors could also be used, including electromagnetic and other detectors which rely on a separate unit to broadcast an orientation field; global position detection circuitry which rely on the satellite global positioning system or local broadcasting devices or a device; Hall Effect sensors; capacitive sensors; and the like.

In specific embodiments, the module will usually be secured to the patient's teeth, typically being removably secured. For example, the module may comprise an enclosure that fits over one or more teeth, allowing the patient to easily insert and remove the module. The alarm generated will typically comprise at least one of sound, vibration, current flow, taste, heat, smell, and the like. In a further aspect of the present invention, an oral appliance for monitoring sleep position comprises an enclosure, circuitry in the enclosure, and an alarm circuit responsive to the circuitry. The enclosure is adapted to be held in a patient's mouth in a fixed orientation relative to the patient's head. The circuitry in the enclosure determines an orientation of the enclosure relative to a field surrounding the enclosure. The alarm circuit is responsive to the circuitry to generate a signal discernible by the patient when the orientation determined by the circuitry corresponds to the patient lying on his or her back, i.e. in a supine position.

In the specific embodiments, the enclosure is adapted to be secured to a patient's teeth, typically in a removable fashion. The enclosure may be adapted to fit over one or more teeth to allow the patient to readily insert and remove the enclosure. For example, the enclosure can be formed as a full or partial structure similar to a retainer or tooth guard formed from vacuum/molded polycarbonate. The fitting and manufacture of such devices is well known in the dental appliance art. The circuitry would then be attached to the retainer structure in a separate housing or module which is attached to the retainer.

The circuitry will most commonly be responsive to a gravitational field and comprise one or more accelerometers. The circuitry will also include a power source, typically a battery, optionally a rechargeable battery or a disposable battery similar to those used in hearing aids.

In addition to circuitry and sensors responsive to a gravitational field, the present invention could employ circuitry responsive to electromagnetic fields, GPS signals, or other broadcast fields which can be sensed in the environment surrounding the patient while the patient sleeps. For example, an electromagnetic field could be generated by a small unit that is placed at the patient's side. GPS signals, in contrast, are available in most areas and are broadcast from an existing satellite network. The alarm circuit generates a signal which is discernible by the patient and preferably non-perceptible to others who may be sleeping near the patient. Generating such a signal in the oral cavity is ideal since a sound or vibration may be transmitted directly to the patient's ears and/or cochlea, for example via the bone conduction. Other signals which are limited or localized to the oral cavity include electrical current which can stimulate the tongue or other oral surfaces, tastes, smells, and the like.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a first embodiment of a patient position monitor in place in a mouth of a sleeping patient.

FIGS. 2A and 2B illustrate the patient position monitor worn by the patient in FIG. 1, where an electronic module is shown removed from an oral appliance in broken line in FIG. 2A.

FIG. 3 illustrates the electronics module of FIGS. 2A and 2B shown in exploded view with an interior circuit board exposed.

FIG. 4 illustrates the patient position monitor of FIGS. 2A and 2B shown with the electronics module removed from the oral appliance, where the electronic module is placed on an inductive charging and communication table, allowing the module to be charged, analyzed and programmed.

FIG. 5 illustrates a patient position monitor having conductive patches on an exterior surface which can transmit an electrical stimulation alarm to the patient.

FIG. 6 illustrates a patient position monitor comprising an oral appliance having the orientation and alarm circuitry embedded into the appliance and sealed from liquid.

FIG. 7 illustrates a patient position monitor comprising an oral appliance in the form of a mouth guard or a retainer which includes rechargeable alarm and position monitoring circuitry attached to an outside surface of the oral application.

FIG. 8 illustrates an oral appliance similar to FIG. 7 except that the alarm and monitoring circuitry is releasably attached to the exterior within an elastic crimping mechanism.

FIGS. 9A and 9C illustrate patient position monitor comprising an oral appliance in the form of a mandibular advancement device or anti-snoring mouth piece having alarm and position monitoring circuitry on a flex circuit which is housed in a hermetically sealed container which is attached to the outside of the oral appliance.

FIGS. 10A and 10B illustrate the flex circuit used in the electronics module of FIGS. 9A and 9C.

FIG. 11 is a schematic circuit diagram showing the components of the alarm and position monitoring circuitry of the present invention.

### DETAILED DESCRIPTION OF THE INVENTION

The present invention provides a patient position monitor including an oral appliance having an electronic module which is capable of determining the orientation of the patient position monitor and providing an alarm when the monitor is in a certain pre-determined orientation. The oral appliance may be a conventional dental or orthodontic appliance, such as a positioner, a retainer, a mandibular advancement device (or other anti-snoring or anti-apnea device), or alternatively may be an appliance specially designed for placement within the oral cavity, particularly over the patient's teeth.

The electronics module typically includes circuitry for both orientation monitoring/determination and for generating the alarm when the orientation falls within predetermined limits, typically when the oral appliance is in a position which would indicate that the patient is in a supine position. The electronics module will be adapted for placement on the oral appliance in the oral cavity and will usually be hermetically sealed to be impervious to saliva in the oral environment for extended periods of time. The electronics module may be attached to an exterior surface of the oral appliance or, alternatively, may be embedded within the appliance itself. In some instances, the oral appliance may serve as the housing or enclosure which defines the electronics module. While the electronics module may be openable to insert batteries, it will more usually be permanently sealed and recharged using an inductive charging base or similar wireless charger. In some instances, the oral appliance may have a cavity, bracket, or other attachment receptacle molded into its structure to allow placement and removal of the electronics module. Exemplary oral appliances include conventional tooth guards and positioners as described in printed Patent Application US 2007/0286448 and US 2010/0006111, the full disclosures of which are incorporated herein by reference.

To use the device, the patient will first turn the electronics module on, typically by pressing an external button on the module but optionally by transmitting a signal from a wand, magnet, or other device. The orientation monitoring electronics will typically be calibrated so that an accelerometer can recognize when the patient is a supine position. Full operation of the device will typically be delayed for an interval to allow a patient to fall asleep. After that interval, the device begins monitoring the patient orientation and detects when the patient assumes the supine position. After a preset threshold or delay interval, typically one minute, two minutes, five minutes, ten minutes or the like, the orientation electronics will initiate the alarm electronics which in turn will produce an alarm to alert the patients that they are on their back and should change position. The alarm will stop after it has detected that the patient has moved away from the supine position, or may be shut off, snoozed, or delayed either by an electronic input, e.g. the button on the device itself or from a remote station.

The patient position monitor will typically be recharged on an inductive recharging surface or station. Optionally, the recharging station can be used to program the device, download a history of events from the device, or both, via a communication link formed through the recharging station which can be attached to a conventional laptop computer or other microprocessor based device. Further optionally, data relating to the patient's sleep position can be selectively or automatically forwarded to a treating physician or other evaluation resource.

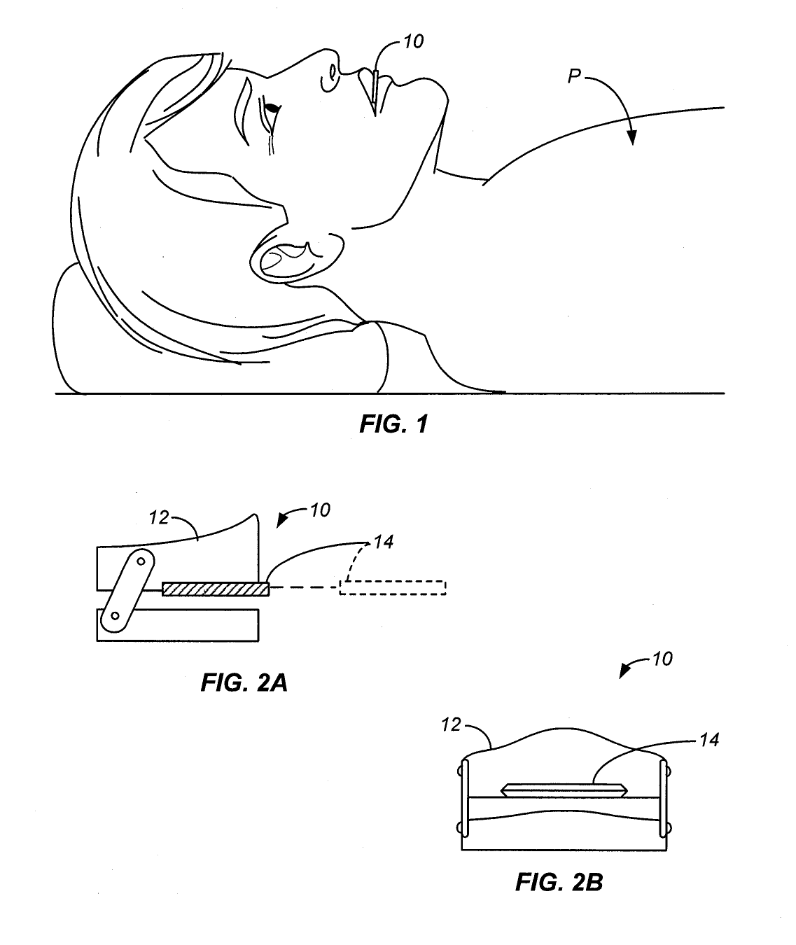

Referring now to FIG. 1, a patient P is shown sleeping in a supine position with a patient position monitor 10 in his mouth. In the position shown, the patient position monitor 10 would begin to alarm after the preset threshold time intervals as set forth above.

Referring now to FIGS. 2A and 2B, it can be seen that the patient position module 10 comprises an oral appliance 12, shown in the form of a mandibular advancement device, and an electronics module 14. The electronics model may be removed from and inserted into the device, as show in FIG. 2A, where the removed module is shown in broken line. When in place, the electronics module 14 will be received in a slot formed in a lower surface of the upper jaw component of the appliance 12.

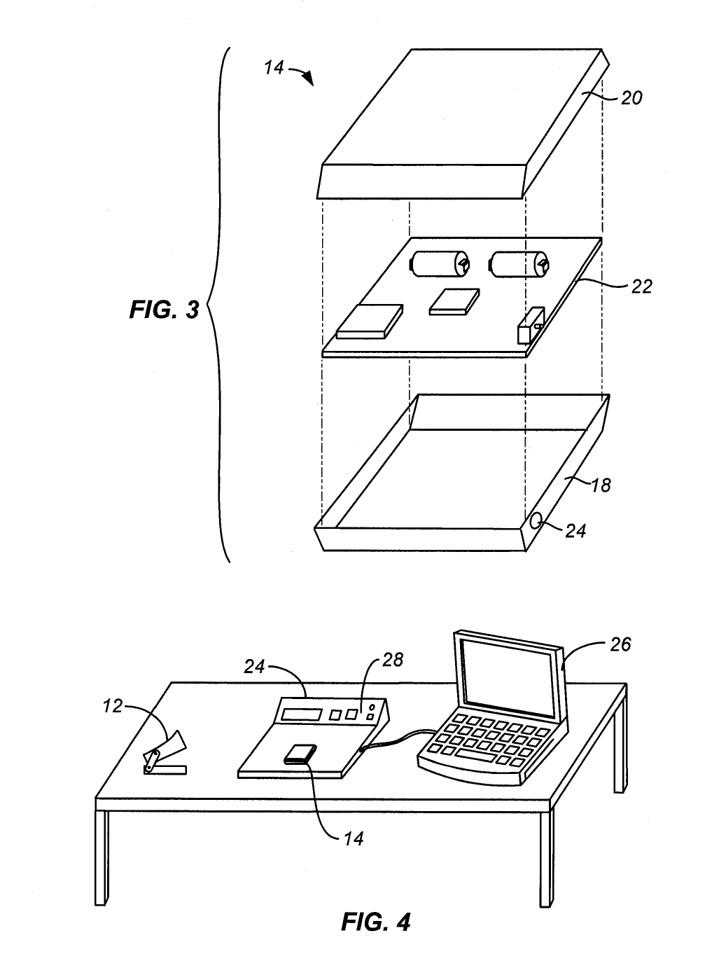

FIG. 3 illustrates the electronics module 14 in an exploded view and including a lower shell 18, an upper shell 20, and an electronic circuit board 22 which is held between the shells. The electronic circuit board 22 includes the electronic components as will be described in more detail below. The shells are hermetically sealed to protect the electronic circuits, and the shells may include a pass-through for sealed button 24, such as an on-and-off button for the electronics module. Other communications and programming may be done through a plug and buss formed through one of the shells, but will more typically be done wirelessly using the induction pad as described previously.

Referring now to FIG. 4, the electronics module 14 may be removed from the appliance and placed on an induction pad 24 which as connected to a laptop computer 26 or other microprocessor based controller. Laptop computer may be used to control the charging and/or reprogramming of the electronics and electronic module. Alternatively, the induction pad 24 may include an on-board interface 28 which allows recharging and/or reprogramming of at least certain parts of the electronics.

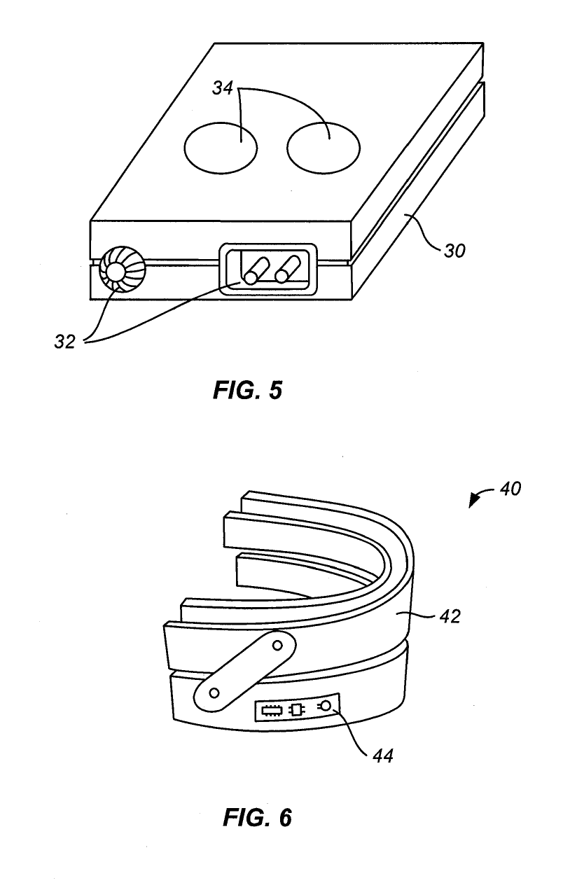

As show in the embodiments thus far, the electronic module 14 will typically include a transducer to generate an audible or vibrational signal and which acts as an alarm to alert the patient to the patient's supine position. As shown in FIG. 5, an alternative electronic module 30 includes some controls and/or communication lines 32 to the exterior. The module 30 also includes exposed external electrodes 34 which are spaced so that they can contact an oral surface, such as the tongue or the palate, when the module is on the oral device and the oral device is in the cavity. Electrodes 34 will be connected to circuits within the module 30 such that a small current is generated when the alarm is triggered. The low current will have a voltage selected to put out a mild sensation to the patient when the current is flowing.

FIG. 6 illustrates an alternative patient position monitor 40 which comprises an oral appliance 42 in the form of a mandibular advancement device. An electronic module 44 is embedded within the lower portion of the oral appliance and is not intended to be removed from the plate. Charging of the battery in the electronic module 44 can be accomplished by placing the entire patient position monitor 40 onto the charging station 24 shown in FIG. 4.

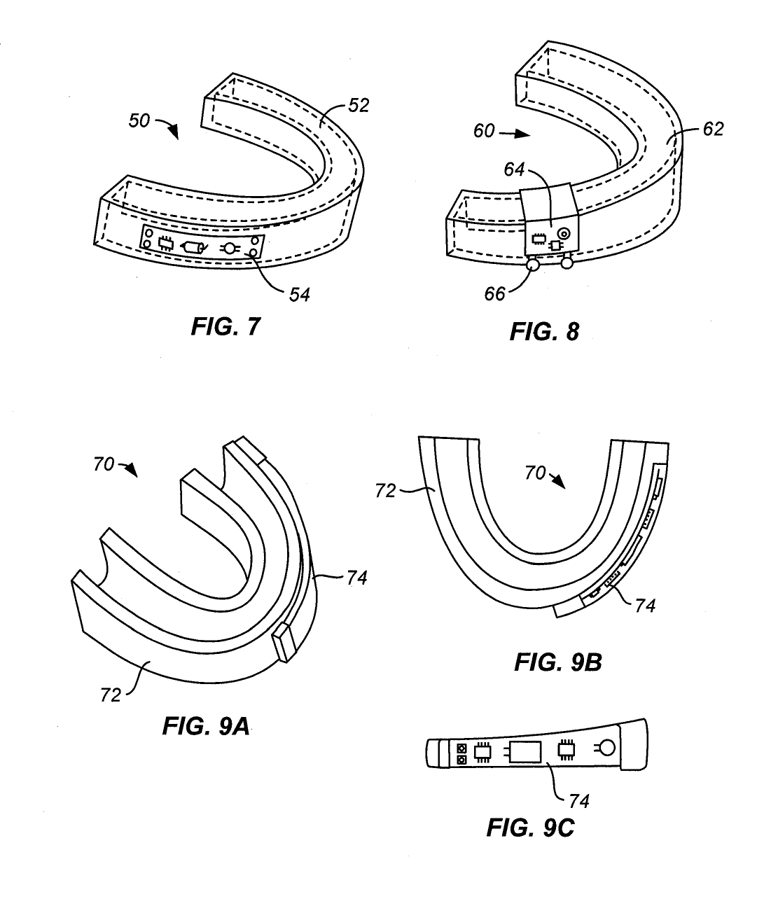

Referring now to FIG. 7, a patient position monitor 50 comprising an oral appliance 52 in the form of a molded retainer or positioner is shown with an electronic module 54 embedded in an outer surface. The electronic module 54 would typically be in the form of a flex circuit and be molded within the appliance 52 to hermitically seal and protect the circuitry.

As shown in FIG. 8, a patient position monitor 60 comprising an oral appliance which is also in the form of a molded retainer or positioner and includes an electronic module 64 which is removably secured over the appliance using an elastic clipping mechanism 66.

Referring now to FIGS. 9A-9C, a patient position monitor 70 comprises a mandibular advisement device 32 and a curved electronic module 74 secured to its outer edge. The components of the electronic module, which typically comprises a flex circuit including all electronic components, are shown in FIG. 9C.

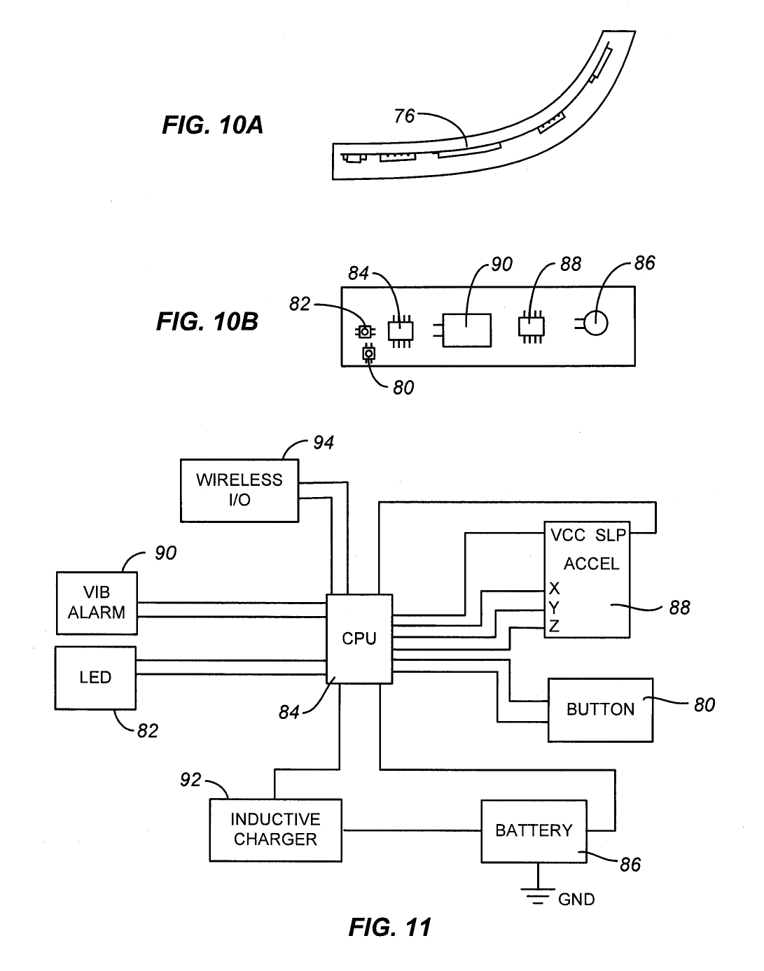

Referring now to FIGS. 10A and 10B, the electronic module 74 may comprise a flex circuit 76 including a button 80, an LED 82, a microprocessor 84, battery 86, accelerometer 88, and a vibrator 90 which serves as the alarm.

Referring to FIG. 11, the interconnections of the circuit components shown in FIGS. 10A and 10B is illustrated. The microprocessor 84 can be an Arduino microprocessor which is connected to on/off button 80, accelerometer 88 (which may be a Freescale MNA7341L or MNA7361L x, y, z-axis accelerometer). The microprocessor is further connected to an LED to indicate whether a system is on or off and to the vibrational transducer. Usually, the circuitry will further comprise an inductive charging component 92 and/or a wireless input-output component 94.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for monitoring sleep position of a patient, said method comprising:
providing a module having an alarm responsive to the module's orientation;
securing the module within the patient's oral cavity;
where the module provides an alarm discernible by the patient when the patient lies on his or her back.

2. A method as in claim 1, wherein the alarm is responsive to changes in the orientation within a gravitational field.

3. A method as in claim 1, wherein the alarm is responsive to changes in a magnetic field.

4. A method as in claim 1, wherein the alarm is responsive to changes in a electrical field.

5. A method as in claim 4, wherein the electrical field is broadcast by a transmitter adjacent to the patient.

6. A method as in claim 1, wherein the alarm is responsive to changes in position relative to a GPS signal.

7. A method as in claim 1, wherein the module is secured to the patient's teeth.

8. A method as in claim 1, wherein the module is removably secured.

9. A method as in claim 8, wherein the module comprises an enclosure that fits over one or more teeth and wherein the patient can manually insert and remove.

10. A method as in claim 9, wherein the alarm comprises at least one of sound, vibration, current, heat, taste and smell.

11. A method as in claim 1, wherein the alarm comprises at least one of sound, vibration, current flow, taste, and smell.

12. A patient position monitor for monitoring sleep position, said oral appliance comprising:
an oral appliance adapted to be held in the patient's mouth in a fixed orientation relative to the patient's head;
orientation circuitry in or attached to the appliance which determines orientation of the appliance relative to a field surrounding the monitor;
an alarm circuit responsive to the orientation circuitry to generate a signal discernible by the patient when the orientation determined by the circuitry corresponds to the patient lying on his or her back.

13. An oral appliance as in claim 12, wherein the enclosure is adapted to be secured to the patient's teeth.

14. An oral appliance as in claim 13, wherein the enclosure is removably securable to the patient's teeth.

15. An oral appliance as in claim 14, wherein the enclosure is adapted to fit over one or more teeth to allow the patient to manually insert and remove the enclosure.

16. An oral appliance as in claim 12, wherein the circuitry is responsive to a gravational field and comprises one or more accelerometers.

17. An oral appliance as in claim 12, wherein the circuitry is responsive to an electromagnetic field.

18. An oral appliance as in claim 12, wherein the circuitry is responsive to an electric field generated by an adjacent transmitter.

19. An oral appliance as in claim 12, wherein the circuitry is responsive to a GPS signal.

20. An oral appliance as in claim 12, wherein the alarm circuit generates a sound signal.

21. An oral appliance as in claim 12, wherein the alarm circuit generates a vibrational signal.

22. An oral appliance as in claim 12, wherein the alarm circuit generates a current flow.

23. An oral appliance as in claim 12, wherein the alarm circuit generates a taste.
